(19) United States
(12) Patent Application Publication
(10) Pub. No.: US 2014/0128726 A1
(43) Pub. Date: May 8, 2014

(54) Alignment of Imaging Modalities

(71) Applicant: MEDTRONIC, INC., Minneapolis, MN (US)

(72) Inventors: Jason Quill, Forest Lake, MN (US); Noah Barka, Minneapolis, MN (US); Jason Bourgoin, Minneapolis, MN (US); Igor Kovalsky, Mounds View, MN (US); Paul Rothstein, Elk River, MN (US)

(73) Assignee: MEDTRONIC, INC., Minneapolis, MN (US)

(21) Appl. No.: 13/668,952

(22) Filed: Nov. 5, 2012

(51) Int. Cl. A61F 2/24; A61B 6/00; A61B 8/13

(52) U.S. Cl. 600/424; 623/2.11

(57) ABSTRACT

Alignment of imaging modalities is disclosed. Orientation of an ultrasound imaging probe can be determined. A fluoroscopic imaging device can be aligned with respect to the orientation of the ultrasound imaging probe and a medical device can be implanted based on images obtained from the ultrasound imaging probe and the fluoroscopic imaging device.

### BACKGROUND

In current surgical and diagnostic medical procedures, multiple types of imaging modalities are utilized to conduct the procedure. For example, a procedure may utilize both a fluoroscopic imaging device (obtaining images using x-ray radiation) and an ultrasound imaging device (obtaining images using sound waves). In each modality, different components can be more or less transparent depending upon the imaging modality. For example, internal tissue can be transparent to a fluoroscopic imaging device, yet visible with an ultrasound imaging device. In other instances, components can create artifacts that disrupt images that are obtained. For example, medical devices such as catheters can be readily observed with a fluoroscopic imaging device. However, such devices can create artifacts when imaged by an ultrasound imaging device.

### SUMMARY

Concepts presented herein relate to aligning imaging modalities. One concept relates to a method of controlling deployment of a prosthetic heart valve having a frame and opposed support arms connected to the frame. The method includes positioning an ultrasound imaging probe at a selected position with respect to a target site including a valve annulus. A probe orientation of the ultrasound imaging probe is sensed with respect to the target site at the selected position and a fluoroscopic imaging device is aligned to an aligned orientation with respect to the probe orientation such that an imaging plane for the ultrasound imaging probe is substantially perpendicular to an imaging plane for the fluoroscopic imaging device. An ultrasound image of the target site is obtained with the ultrasound imaging probe in the selected position. A fluoroscopic image of the target site is obtained with the fluoroscopic imaging device in the aligned orientation. The valve orientation and a valve position of the support arms with respect to the valve annulus are determined as a function of the ultrasound image and the fluoroscopic image.

Another concept relates to a system having a fluoroscopic imaging device mounted to a support head maintaining the fluoroscopic imaging device in a selected orientation. The system also includes an ultrasound imaging probe including an accelerometer measuring a probe orientation of the ultrasound imaging probe with respect to gravity. A processor is coupled to the accelerometer and provides a signal indicative of the probe orientation relative to the selected orientation.

In yet a further concept, a delivery device is disclosed for percutaneously deploying a stented prosthetic heart valve including a stent frame to which a valve structure is attached. The device includes a delivery sheath assembly terminating at a distal end and defining a lumen. The device further includes an inner shaft slideably disposed within a lumen and in contact with the stent frame. An accelerometer provides a signal indicative of an orientation of the stent frame with respect to gravity. The device is configured to provide a loaded state in which the delivery sheath assembly retains the stented prosthetic heart valve over the inner shaft and a deployment state in which the distal end of the delivery sheath assembly is withdrawn from the prosthetic heart valve to permit the prosthetic heart valve to release from the inner shaft assembly.

In yet a further concept, a delivery device for deploying an implantable medical device visible under fluoroscopy is provided. The delivery device includes a shaft assembly and an accelerometer coupled to the shaft assembly to provide a signal indicative of an orientation of the shaft assembly.

### BRIEF DESCRIPTION OF THE DRAWINGS

FIG. 1A is a schematic diagram of a system for performing a procedure using multiple imaging modalities.

FIG. 1B is a flow diagram of a method for aligning multiple imaging modalities during a surgical procedure.

FIG. 2A is a side view of a prosthetic valve in a first, expanded arrangement.

FIG. 2B is a side view of the prosthetic valve of FIG. 2A in a second, compressed arrangement.

FIG. 3A is an isometric view of an exemplary delivery system including a delivery device configured to deliver a prosthetic valve to a target site.

FIG. 3B is an exploded view of the delivery device illustrated in FIG. 3A.

FIG. 4 is a schematic diagram of a native mitral valve annulus.

FIGS. 5-7 are schematic sectional views of a heart illustrating implantation of a prosthetic valve.

FIG. 8 is a flow diagram of a method for implanting a prosthetic valve.

### DETAILED DESCRIPTION

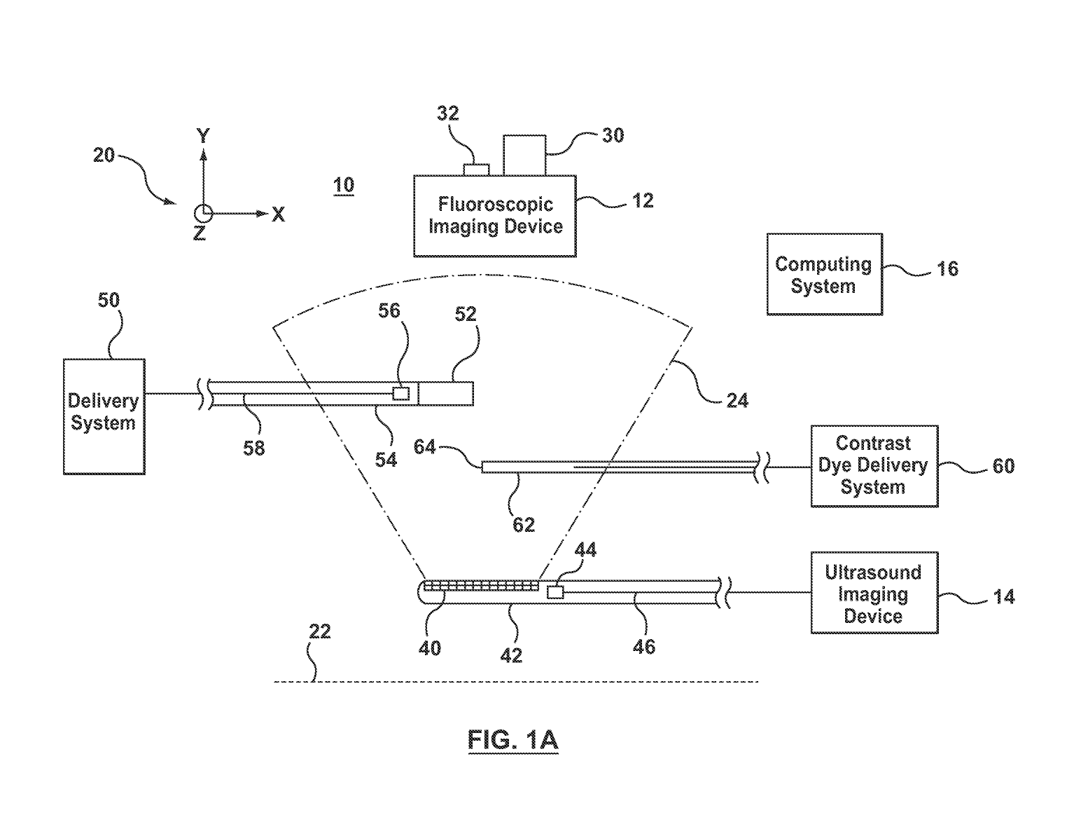

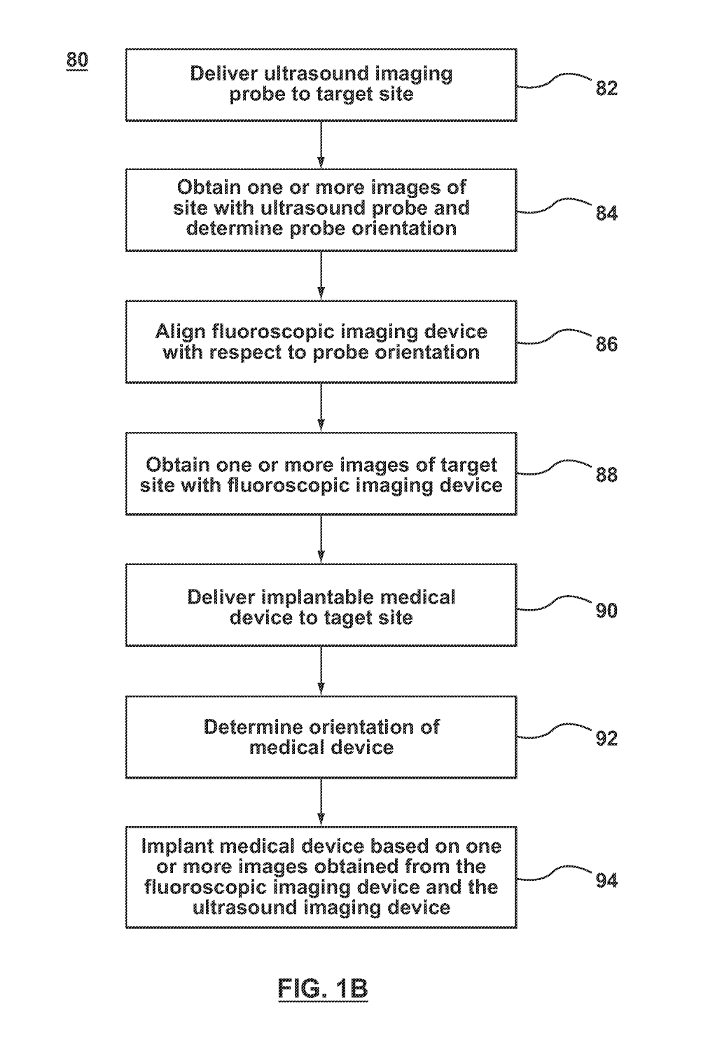

FIG. 1A is a schematic diagram of a system 10 including a fluoroscopic imaging device 12 and an ultrasound imaging device 14. The fluoroscopic imaging device 12 and ultrasound imaging device 14 are each coupled with a suitable computing system 16. The computing system 16 can be equipped to display images captured by the fluoroscopic imaging device 12 and the ultrasound imaging device 14.

The fluoroscopic imaging device 12 and ultrasound imaging device 14 capture images with respect to a Cartesian coordinate system 20. As illustrated, the coordinate system 20 includes three orthogonal axes (denoted X, Y and Z axes), which define three orthogonal planes, namely an XY plane, an XZ plane and a YZ plane. In the embodiment illustrated, coordinate system 20 is coincident with gravity such that the Y axis is coaxial with the gravitational pull on system 10. In FIG. 1, fluoroscopic imaging device 12 is positioned to capture images coincident with an imaging plane 22, which is co-planar with the XZ plane of the coordinate system 20. In a similar manner, ultrasound imaging device 14 obtains images along a plane 24 that is co-planar with the XY plane of coordinate system 20. As will be discussed in more detail below, system 10 is equipped to determine an orientation for the imaging planes 22 and 24 and selectively adjust orientation of the planes 22 and 24 to a desired alignment.

As schematically illustrated in FIG. 1A, the fluoroscopic imaging device 12 can be mounted to a support head 30 capable of adjusting a position and orientation of the fluoroscopic imaging device 12 within the coordinate system 20. In particular, the support head 30 can position and orient the device 12 to align its corresponding imaging plane 22. To this end, an accelerometer 32 can be coupled with the fluoroscopic imaging device 12 to provide an indication of orientation of the device 12 with respect to coordinate system 20. In particular, accelerometer 32 measures the orientation of device 12 with respect to gravity. As coordinate system 20 is coincident with gravity, the orientation of device 12 within coordinate system 20 can be determined by the accelerometer 32. An output signal of the accelerometer 32 providing an indication of the orientation of fluoroscopic imaging device 12 can be provided to the computing system 16 through a suitable connection (wired or wireless).

In the embodiment illustrated in FIG. 1A, the ultrasound imaging device 14 includes an ultrasound imaging probe 40 positioned at a distal end of a suitable catheter 42. In one embodiment, the catheter 42 is inserted into a patient and positioned such that the imaging probe 40 can obtain images of an internal portion of the patient. In one example, the imaging probe is an echocardiography probe configured to image one or more chambers of a patient's heart. When positioned during an echocardiogram, probe 40 can capture images of the patient's heart and one is able to evaluate operation of the heart as well as view the valves providing blood flow through the patient's circulatory system. An accelerometer 44 can be coupled to the catheter 42 in order to provide an indication of the orientation of imaging probe 40 with respect to gravity. The accelerometer 44 can be communicatively coupled with the computing system 16. In one embodiment, wiring 46 leads from the accelerometer 44 along catheter 42 to a suitable connection (wired or wireless) with computing system 16.

System 10 further includes a delivery system 50 configured to deliver an implantable medical device 52 using a suitable catheter 54 to a target site. An accelerometer 56 is coupled with the catheter 54 to provide an indication of the orientation of the device 52 with respect to coordinate system 20. Device 52 and catheter 54 create artifacts when imaged by imaging probe 40, yet are discernable in images obtained by the fluoroscopic imaging device 12. To that end, in one embodiment, the device 52 and/or catheter 54 may include radiopaque material to enhance the visibility of the device 52 and/or catheter 54. Wiring 58 can be used to couple the accelerometer 56 to a suitable connection (wired or wireless) with computing system 16.

Further still, system 10 may include a contrast dye delivery system 60 having a catheter 62. Catheter 62 can deliver and inject a dye (e.g., a radiopaque dye) into a patient that is visible to the fluoroscopic imaging device 12. In particular, the catheter 62 includes a lumen with an outlet 64 in which dye exits from the catheter 62. The dye can be injected into a fluid stream such that the device 12 can determine flow of fluid within a patient.

Given components of system 10 above, one environment for which the system 10 is useful is in a procedure where a surgeon implants medical device 52 within a patient. In such a procedure, internal tissue of the patient can generally be visible with the ultrasound imaging probe 40. Moreover, the delivery system 50 and contrast dye delivery system 60 are visible to the fluoroscopic imaging device 12. The accelerometers 32, 44 and 56 can be utilized in order to align the fluoroscopic imaging device 12, the probe 40 and the implantable device 52 so as to properly implant the device 52 with respect to tissue within a patient. The accelerometers 32, 44 and 56 can take various forms, such as being embodied as single axis or triaxial accelerometer and can be formed of various components such as piezoelectric, piezoresistive and capacitive components.

FIG. 1B is a flow diagram of an exemplary method 80 for positioning an implantable medical device 52 using system 10 of FIG. 1A. At step 82, the ultrasound imaging probe 40 is delivered to a target site. In particular, the catheter 42 is introduced within a patient and maneuvered to a desired position with a probe orientation so as to image the target site. The desired position can be selected so as to provide a surgeon with a selected view of the target site with a particular alignment, where imaging plane 24 is oriented such that suitable images can be captured by probe 40. At step 84, one or more images of the target site are obtained with the ultrasound imaging probe and the probe orientation is determined with the probe accelerometer 44. In one embodiment, probe 40 can be maintained in the desired position such that a surgeon can continuously obtain ultrasound images so as to monitor the target site with the probe 40 during the procedure to implant device 52.

Based on the orientation of probe 40 as calculated by the accelerometer 44, fluoroscopic imaging device 12 can be aligned with respect to the probe orientation at step 86, for example using support head 30 and accelerometer 32. In one embodiment, imaging device 12 is aligned such that the corresponding imaging plane 22 is orthogonal to imaging plane 24 of ultrasound probe 40. As such, a user can easily compare images from separate modalities (i.e., images from the fluoroscopic imaging device and the ultrasound imaging device) to evaluate the target site and correctly position the device 52 during implantation. In a further embodiment, fluoroscopic imaging device 12 can be aligned based on images obtained by the probe 40. For example, in one embodiment, the probe 40 can include an array that is substantially rectangular when viewed from a selected orientation. The device 12 can thus be oriented to produce a rectangular representation of probe 40. As such, this technique can be used to confirm a desired orientation of device 12. At step 88, one or more fluoroscopic images are obtained using the fluoroscopic imaging device 12. As such, tissue viewable by probe 40 and the device 52 viewable by imaging device 12 can be aligned with reference to images obtained using both probe 40 and imaging device 12. In one embodiment, device 12 can continuously provide images such that a surgeon can monitor the target site.

At step 90, the implantable device 52 is delivered to the target site using the delivery system 50. Once positioned at the target site, the orientation of the implantable device 52 can be determined at step 92 using accelerometer 56. Based on one or more of the images obtained from the ultrasound imaging probe and the fluoroscopic imaging device, the implantable device 52 can be implanted at step 94. Method 80 can be modified as desired. For example, the contrast dye delivery system 60 can inject dye one or more times during method 80 as desired. Additionally, the target site can be evaluated after implantation to confirm proper operation of the device 52.

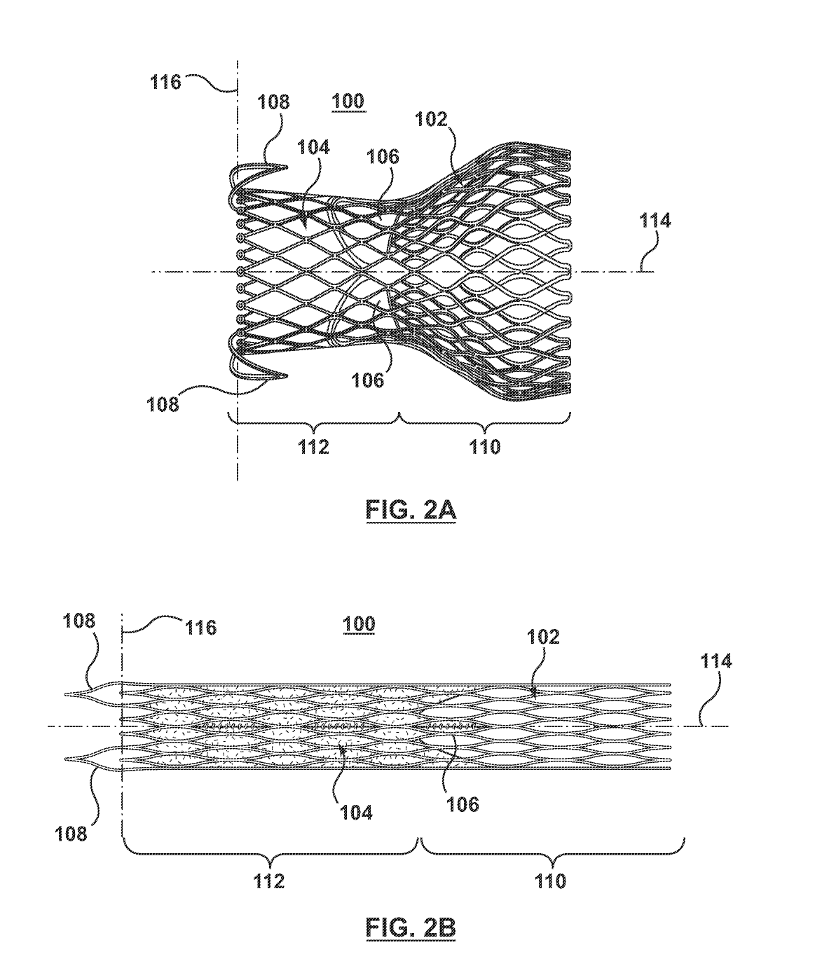

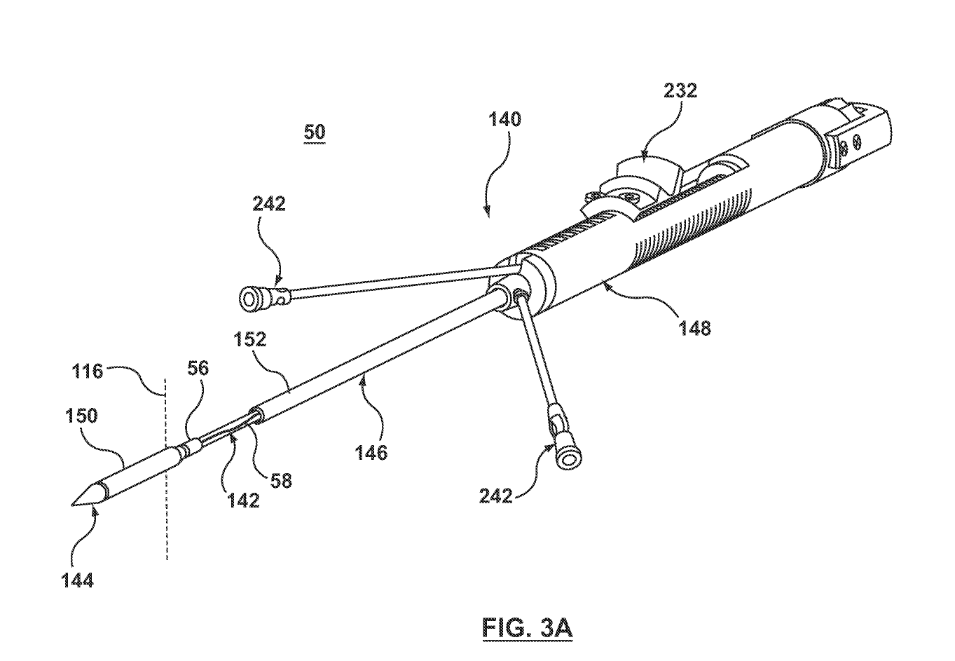

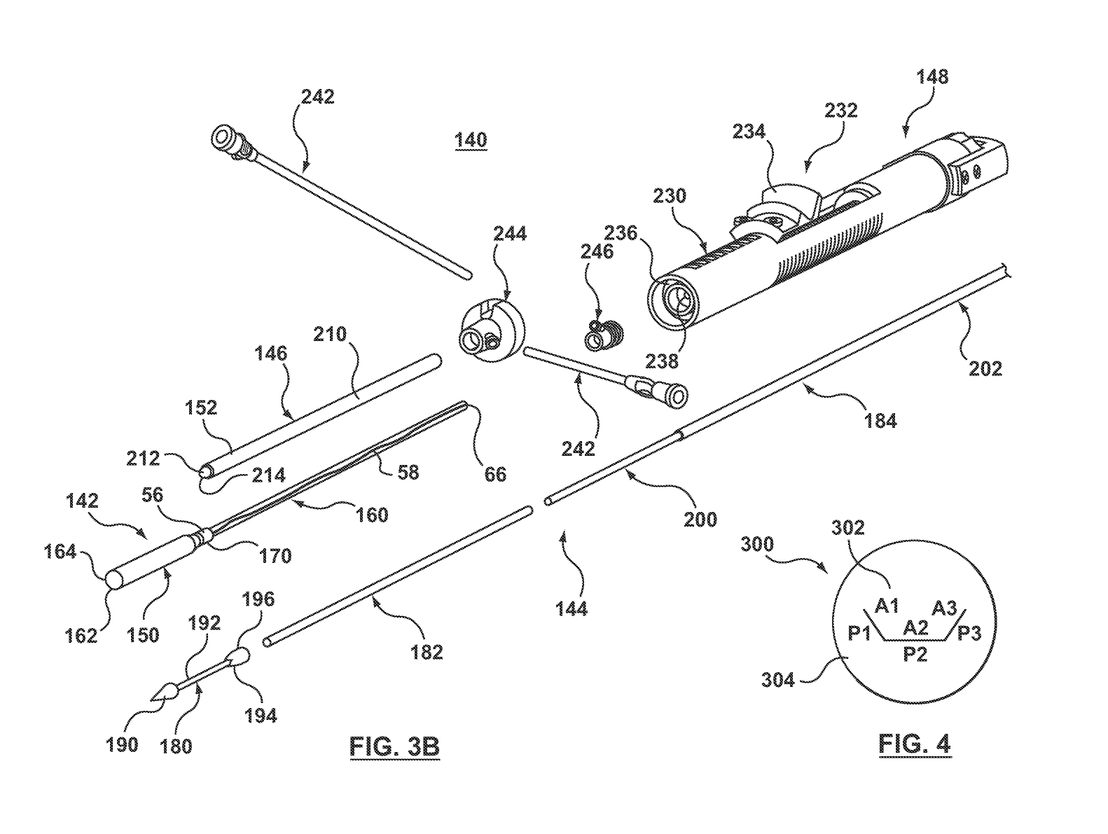

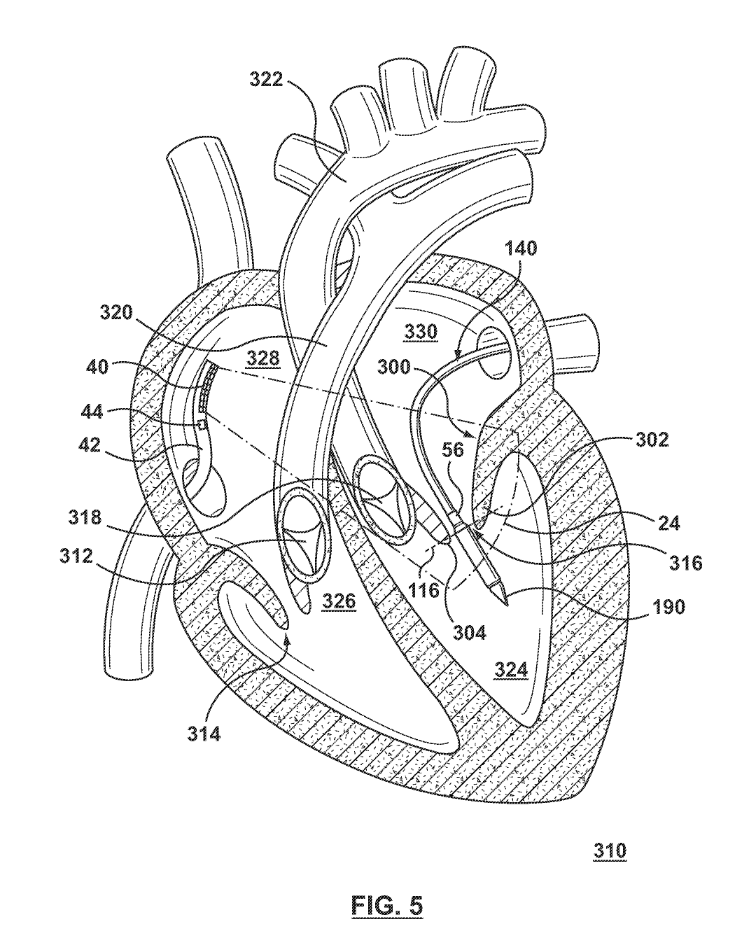

With the above understanding of aligning imaging modalities in mind, the concepts presented herein can be applied in various situations when aligning one or more medical devices with tissue internal to a patient. For example, a catheter delivered occluder that blocks a hole in a wall of a heart can be used within the system 10 of FIG. 1A. Further still, aneurysm repair techniques can be utilized with system 10, such as to implant a graft and/or repair an aneurysm. One particular example medical procedure using concepts presented herein includes implantation of a stented prosthetic valve. In this procedure, implantable medical device 52 is embodied as a prosthetic valve and is compacted for delivery in delivery device 50 (embodied as a catheter-based delivery device) and then advanced, for example, through an opening in the femoral artery and through the aorta to the heart, where the prosthetic valve is then deployed in the annulus of the valve to be repaired (e.g., the mitral valve annulus). As discussed below, FIGS. 2A and 2B illustrate an exemplary a prosthetic valve, whereas FIGS. 3A and 3B illustrate an exemplary delivery device for delivering the prosthetic valve to a target site.

As referred to herein, stented transcatheter prosthetic heart valves useful with and/or as part of the various systems, devices, and methods of the present disclosure may assume a wide variety of different configurations, such as a bioprosthetic heart valve having tissue leaflets or a synthetic heart valve having polymeric, metallic, or tissue-engineered leaflets, and can be specifically configured for replacing any heart valve. Thus, the stented prosthetic heart valve useful with the systems, devices, and methods of the present disclosure can be generally used for replacement of a native aortic, mitral, pulmonic, or tricuspid valve, for use as a venous valve, or to replace a failed bioprosthesis, such as in the area of an aortic valve or mitral valve, for example.

With the above understanding in mind, one non-limiting example of a stented prosthetic heart valve 100 useful with systems and methods of the present disclosure is illustrated in FIG. 2A. As a point of reference, the prosthetic heart valve 100 is shown in a normal or expanded arrangement in the view of FIG. 2A; FIG. 2B illustrates the prosthetic heart valve 100 in a compressed arrangement (e.g., when compressively retained within an outer catheter or sheath). The prosthetic heart valve 100 includes a stent or stent frame 102 and a valve structure 104.

The stent frame 102 is generally constructed so as to be self-expandable from the compressed arrangement (FIG. 2B) to the normal, expanded arrangement (FIG. 2A). In other embodiments, the stent frame 102 is expandable to the expanded arrangement by a separate device (e.g., a balloon internally located within the stent frame 102). The valve structure 104 is assembled to the stent frame 102 and provides two or more leaflets 106. The valve structure 104 can assume any of the forms described above, and can be assembled to the stent frame 102 in various manners, such as by sewing the valve structure 104 to one or more of the wire segments defined by the stent frame 102. First and second opposed support arms 108 are positioned at an end of the stent frame 102 and configured to capture native leaflets of a mitral valve as discussed below in the expanded arrangement of FIG. 2A. In the compressed arrangement of FIG. 2B, the support arms 108 are folded to extend in a direction away from the stent frame 102. Upon release from compressive forces retaining the valve in the compressed arrangement of FIG. 2B, support arms 108 will fold back upon the stent frame 102 to transition to the expanded arrangement.

Given the components of the prosthetic heart valve 100, the valve can be defined to include an inflow section 110 (receiving fluid) and an outflow section 112 (forcing out fluid). Moreover, the stent frame 102 defines a central axis 114 extending in a direction from the inflow section 110 to the outflow section 112 and a support arm axis 116 delineating an orientation of the support arms 108.

With the but one acceptable construction of FIGS. 2A and 2B, the prosthetic heart valve 100 is configured for repairing a mitral valve. Alternatively, other shapes are also envisioned, adapted to the specific anatomy of the valve to be repaired (e.g., stented prosthetic heart valves in accordance with the present disclosure can be shaped and/or sized for replacing a native mitral, aortic, pulmonic, or tricuspid valve). With the one construction of FIGS. 2A and 2B, the valve structure 104 extends less than the entire length of the stent frame 102, but in other embodiments can extend along an entirety, or a near entirety, of a length of the stent frame 102. A wide variety of other constructions are also acceptable and within the scope of the present disclosure. For example, the stent frame 102 can have a more cylindrical shape in the normal, expanded arrangement.

Given the above description of the stented prosthetic heart valve 100, one embodiment of delivery system 50 for repairing a defective heart valve is shown in FIG. 3A, and includes a delivery device 140 for percutaneously delivering and implanting the prosthetic heart valve 100. The delivery device 140 includes a delivery sheath assembly 142, an inner shaft assembly 144 (referenced generally), an outer stability tube 146, and a handle 148. Accelerometer 56 and wiring 58 are coupled to the delivery sheath assembly 142 to measure an orientation of prosthetic heart valve, which is hidden from view in FIG. 3A. During loading of the valve, support arms axis 116 can be aligned with the accelerometer 56 such that the orientation of axis 116 (and thus orientation of the valve 100) can be determined during deployment of the valve.

In general terms, the system 50 is transitionable from a loaded or delivery condition (shown in FIG. 3A) in which the stented prosthetic heart valve is contained within a capsule 150 of the delivery sheath assembly 142, to a deployed condition in which the capsule 150 is retracted from the prosthetic heart valve, thereby permitting the prosthetic heart valve to self-expand (or alternatively be caused to expand by a separate mechanism such as a balloon) and release from the delivery device 140. As part of this transitioning, the delivery sheath assembly 142 is slidable relative to the stability tube 146, with the stability tube 146 serving to frictionally isolate the delivery sheath assembly 142 from a separate introducer device (not shown). Further, a distal region 152 of the stability tube 146 has an expandable or stretchable attribute adapted to readily receive the capsule 150 when transitioning from a delivery state to a deployed state. With this construction, the stability tube 146 can be closely positioned to the capsule 150 during delivery and deployment, thereby desirably enhancing stabilization of the delivery sheath assembly 142.

Components in accordance with some embodiments of the delivery device 140 are shown in greater detail in FIG. 3B. As a point of reference, various features of the components 142-148 reflected in FIG. 3B and described below can be modified or replaced with differing structures and/or mechanisms. Thus, the present disclosure is in no way limited to the delivery sheath assembly 142, the inner shaft assembly 144, the handle 148, etc., shown and described below.

In some embodiments, the delivery sheath assembly 142 includes the capsule 150 and a shaft 160, and defines a lumen 162 (referenced generally) extending from a distal end 164 to a proximal end 166. In some constructions, the capsule 150 and the shaft 160 are comprised of differing materials and/or constructions, with the capsule 150 having a longitudinal length approximating (e.g., slightly greater than) a length of the prosthetic heart valve 100 (FIG. 2B) to be used with the device 140. The capsule 150 is attached to, and extends distally from, the shaft 160 and in some embodiments has a more stiffened construction (as compared to a stiffness of the shaft 160) that exhibits sufficient radial or circumferential rigidity to overtly resist the expected expansive forces of the stented prosthetic heart valve 100 when compressed within the capsule 150. For example, the shaft 160 can be a polymer tube embedded with a metal braiding, whereas the capsule 150 includes a laser-cut metal tube that is optionally embedded within a polymer covering. Alternatively, the capsule 150 and the shaft 160 can have a more uniform construction (e.g., a continuous polymer tube).

Regardless, the capsule 150 is constructed to compressively retain the stented prosthetic heart valve 100 at a predetermined diameter when loaded within the capsule 150, and the shaft 160 serves to connect the capsule 150 with the handle 148. To better accommodate a size of the compressed prosthesis 100 while at the same time maintaining an overall low profile, an outer diameter of the capsule 150 can be greater than an outer diameter of the shaft 160 in some embodiments, with the resultant construction providing the capsule 150 with a discernable proximal end 170. The shaft 160 (as well as the capsule 150) is constructed to be sufficiently flexible for passage through a patient's vasculature, yet exhibits sufficient longitudinal rigidity to effectuate desired axial movement of the capsule 150. In other words, proximal retraction of the shaft 160 is directly transferred to the capsule 150 and causes a corresponding proximal retraction of the capsule 150. In other embodiments, the shaft 160 is further configured to transmit a rotational force or movement onto the capsule 150.

The inner shaft assembly 144 can have various constructions appropriate for supporting a stented prosthetic heart valve within the capsule 150. For example, the inner shaft assembly 144 can include a retention member 180, an intermediate tube 182, and a proximal tube 184. In general terms, the retention member 180 is akin to a plunger, and incorporates features for retaining the stented prosthetic heart valve 100 (FIG. 2B) within the capsule 150 as described below. The intermediate tube 182 connects the retention member 180 to the proximal tube 184, with the proximal tube 184, in turn, coupling the inner shaft assembly 144 with the handle 148. The components 180-184 can combine to define a continuous lumen 186 (referenced generally) sized to slidably receive an auxiliary component such as a guide wire (not shown).

The retention member 180 can include a tip 190, a support tube 192, and a hub 194. The tip 190 forms or defines a nose cone having a distally tapering outer surface adapted to promote atraumatic contact with bodily tissue. The tip 190 can be fixed or slidable relative to the support tube 192. The support tube 192 extends proximally from the tip 190 and is configured to internally support a compressed, stented prosthetic heart valve generally disposed thereover, and has a length and outer diameter corresponding with dimensional attributes of the prosthetic heart valve. The hub 194 is attached to the support tube 192 opposite the tip 190 (e.g., adhesive bond) and provides a coupling structure 196 (referenced generally) configured to selectively capture a corresponding feature of the prosthetic heart valve.

The coupling structure 196 can assume various forms, and is generally located along an intermediate portion of the inner shaft assembly 144. In some embodiments, the coupling structure 196 includes one or more fingers sized to be slidably received within corresponding apertures formed by the prosthetic heart valve stent frame 102 (FIG. 2A). For example, the stent frame 102 can form wire loops at a proximal end thereof that are releasably received over respective ones of the fingers when compressed within the capsule 150. Other releasable coupling arrangements are also acceptable, such as the hub 194 forming one or more slots sized to slidably receive a corresponding component(s) of the prosthetic heart valve (e.g., a bar or leg segment of the stent frame). Further, the inner shaft assembly 144 can incorporate additional structures and/or mechanisms that assist in temporarily retaining the prosthetic heart valve (e.g., a tubular segment biased over the coupling structure 196.

The intermediate tube 182 is formed of a flexible material (e.g., PEEK), and is sized to be slidably received within the delivery sheath assembly 142 and in particular the shaft 160. The proximal tube 184 can include a leading portion 200 and a trailing portion 202. The leading portion 200 serves as a transition between the intermediate and proximal tubes 182, 184, and thus can be a flexible tubing (e.g., PEEK) having a diameter slightly less than that of the intermediate tube 182. The trailing portion 202 has a more rigid construction, configured for robust assembly with the handle 148. For example, the trailing portion 202 can be a metal hypotube, although other constructions are also acceptable. In yet other embodiments, the intermediate and proximal tubes 182, 184 are integrally formed as a single, homogenous tube or solid shaft.

The stability tube 146 includes or defines the distal region 152 and a proximal region 210. The stability tube 146 forms a lumen 212 (referenced generally) sized to be slidably received over the delivery sheath assembly 142 as described below, with the stability tube 146 terminating at a distal end 214.

The proximal region 210 connects the distal region 152 with the handle 148. With this construction, the stability tube 146 serves as a stability shaft for the delivery sheath assembly 142, and has a length selected to extend over a significant (e.g., at least a majority), and in some embodiments at least 80%, of a length of the delivery sheath assembly 142 in distal extension from the handle 148. Further, the stability tube 146 exhibits sufficient radial flexibility to accommodate passage through a patient's vasculature (e.g., the femoral artery and the aortic arch).

The handle 148 generally includes a housing 230 and one or more actuator mechanism 232 (referenced generally). The housing 230 maintains the actuator mechanism 232, with the handle 148 configured to facilitate sliding movement of the delivery sheath assembly 142 relative to the inner shaft assembly 144 and the stability tube 146. The housing 230 can have any shape or size appropriate for convenient handling by a user. In one simplified construction of the actuator mechanism 232, a user interface or actuator 234 is slidably retained by the housing 230 and coupled to a connector body 236. The inner shaft assembly 144, and in particular the proximal tube 184 is slidably received within a passage 238 (referenced generally) of the connector body 236 and is rigidly coupled to the housing 230. With this but one acceptable construction, the deployment actuator 234 can be operated by a user to effectuate axial or longitudinal movement of the delivery sheath assembly 142 relative to the inner shaft assembly 144 and the stability tube 146. In some embodiments, the housing 230 can further incorporate a second actuator mechanism (not shown) that facilitates user-actuated movement of the stability tube 146 relative to the delivery sheath assembly 142. Further, the handle 148 can include other features, such as optional port assemblies 242, a cap 244, and/or a manifold 246 as shown.

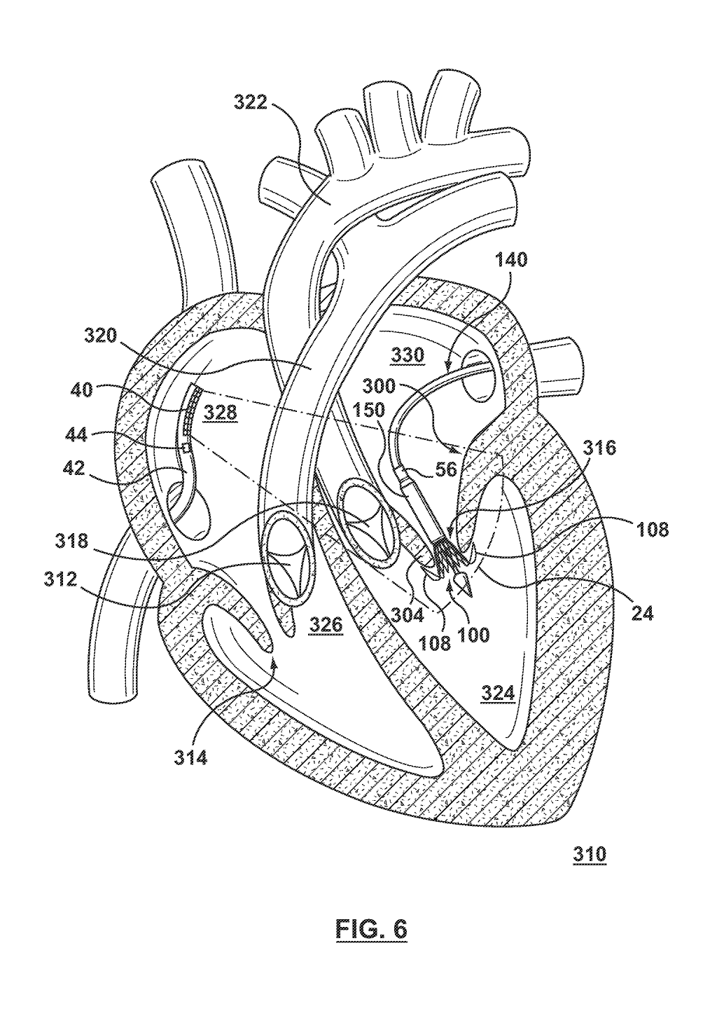

Delivery system 50 and valve 100 are useful in replacing a native mitral valve of a patient. In one embodiment, replacement of a native mitral valve includes capturing native leaflets and deploying the valve within the mitral valve annulus. FIG. 4 is a schematic view of a mitral valve annulus 300 including an anterior leaflet 302 and a posterior leaflet 304. The anterior leaflet 302 includes regions A1, A2 and A3. Similarly, the posterior leaflet 304 includes posterior regions P1, P2 and P3 adjacent regions A1, A2 and A3 of anterior leaflet 302. During deployment of valve 100, it is important for the support arms 108 of the prosthetic heart valve 100 to capture the anterior leaflet 302 at the A2 region and capture the posterior leaflet 304 at the P2 region. Once the support arms 108 capture the leaflets 302 and 304, the valve 100 can be finally deployed. In order to properly position the support arms 108 to capture leaflets 302 and 304 at the A2 and P2 regions, alignment of ultrasound imaging probe 40 and fluoroscopic imaging device 12 assists in the ability to capture the leaflets 302 and 304 with support arms 108 during deployment of valve 100, as will be discussed below.

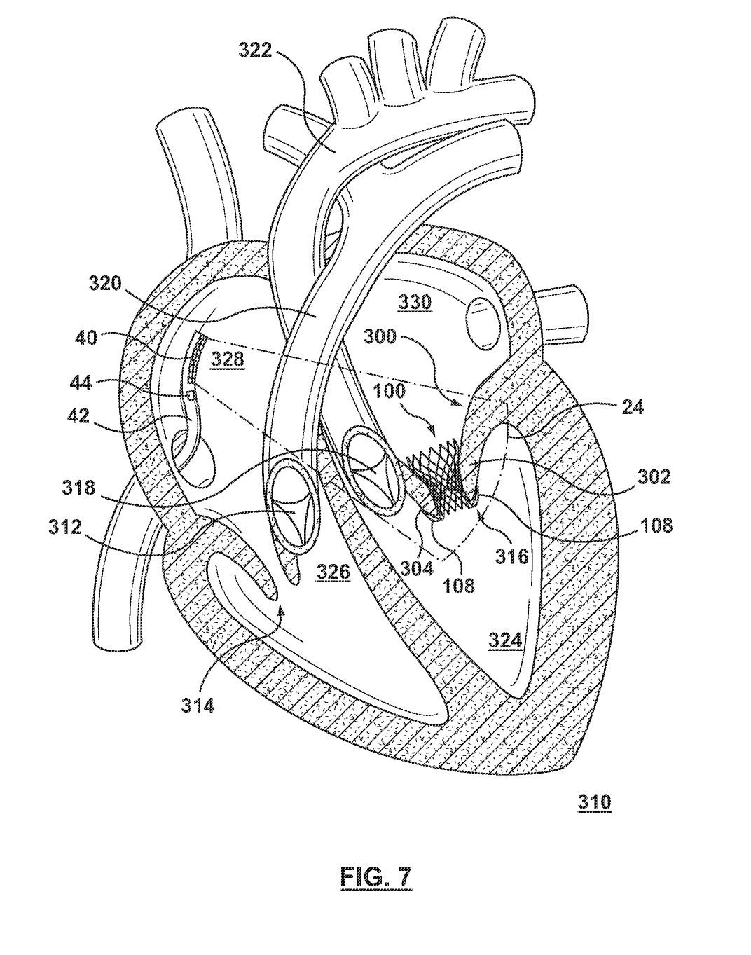

FIGS. 5-7 are successive views illustrating implantation of valve 100 within valve annulus 300. By way of understanding, FIGS. 5-7 illustrate a schematic representation of the interior of human heart 310. Human heart 310 includes four valves that work in a synchrony to control the flow of blood through the heart. Tricuspid valve 314, situated between the right atrium 328 and right ventricle 326, and mitral valve 316, between left atrium 380 and left ventricle 324 facilitate filling of ventricles 326 and 324 on the right and left sides, respectively, of heart 310. Aortic valve 318 is situated at the junction between aorta 322 and left ventricle 324 and facilitates blood flow from heart 310 through aorta 322 to the peripheral circulation. Pulmonary valve 312 is situated at the junction of right ventricle 326 and pulmonary artery 320 to the lungs for oxygenation.

The four valves work by opening and closing in harmony with each other. During diastole, tricuspid valve 314 and mitral valve 316 open and allow blood flow into ventricles 326 and 322, and the pulmonic valve and aortic valve are closed. During systole, aortic valve 318 and pulmonary valve 322 open and allow blood flow from left ventricles 324 and right ventricle 326 into aorta 322 and pulmonary artery 320, respectively.

As illustrated in FIG. 5, the ultrasound catheter 42 has positioned ultrasound imaging probe 40 in the right atrium 328, so as to image the mitral valve annulus 300. The valve delivery device 140 has been delivered through the left atrium 330 and into the left ventricle 324 through the mitral valve 316. Imaging plane 24 is positioned with respect to mitral valve 316 so as to obtain a sectional view of leaflets 302 and 304, particularly at the A2 and P2 regions. Delivery sheath capsule 150 has been deployed through the mitral valve 316 to the left ventricle 324. Although not shown for clarity purposes, dye delivery system 60 can also be delivered to heart 310 and utilized as desired while obtaining images with fluoroscopic imaging device 12.

Using accelerometer 44, an orientation of the probe 40 can be determined with respect to gravity, thus providing an indication of the orientation of imaging plane 24. Based on the probe orientation, the fluoroscopic imaging device 12 can be aligned using accelerometer 30 such that imaging plane 22 and imaging plane 24 are orthogonal to one another. With the imaging planes 22 and 24 in a desired alignment, images obtained with fluoroscopic imaging device 12 can be directly comparable with images obtained with probe 40. Using accelerometer 56, a device orientation is obtained and can be adjusted for implantation of valve 100. In particular, support arm axis 116 can be oriented to also be orthogonal to imaging plane 24, such that leaflets 302 and 304 can be captured.

In FIG. 6, the delivery sheath capsule 150 has been retracted so as to partially expose the support arms 108 of valve 100. In particular, support arms 108 begin to fold over such that leaflets 302 and 304 are captured with arms 108. The ultrasound imaging probe 40 can then be utilized to determine if the support arms 108 have indeed captured the valve leaflets 302 and 304. For example, the leaflets 302 and 304 having shown little or no movement would indicate capture by support arms 108. If the ultrasound imaging probe indicates that indeed the leaflets 302 and 304 have been captured by the support arms 108, the delivery sheath capsule 150 can be retracted fully in order to completely deploy the heart valve within the mitral valve annulus 300.

FIG. 7 illustrates the valve fully deployed within the mitral valve annulus 300. At this point, fluoroscopic imaging device 12 and/or ultrasound imaging device 14 can be utilized to evaluate whether the valve 100 is operating properly.

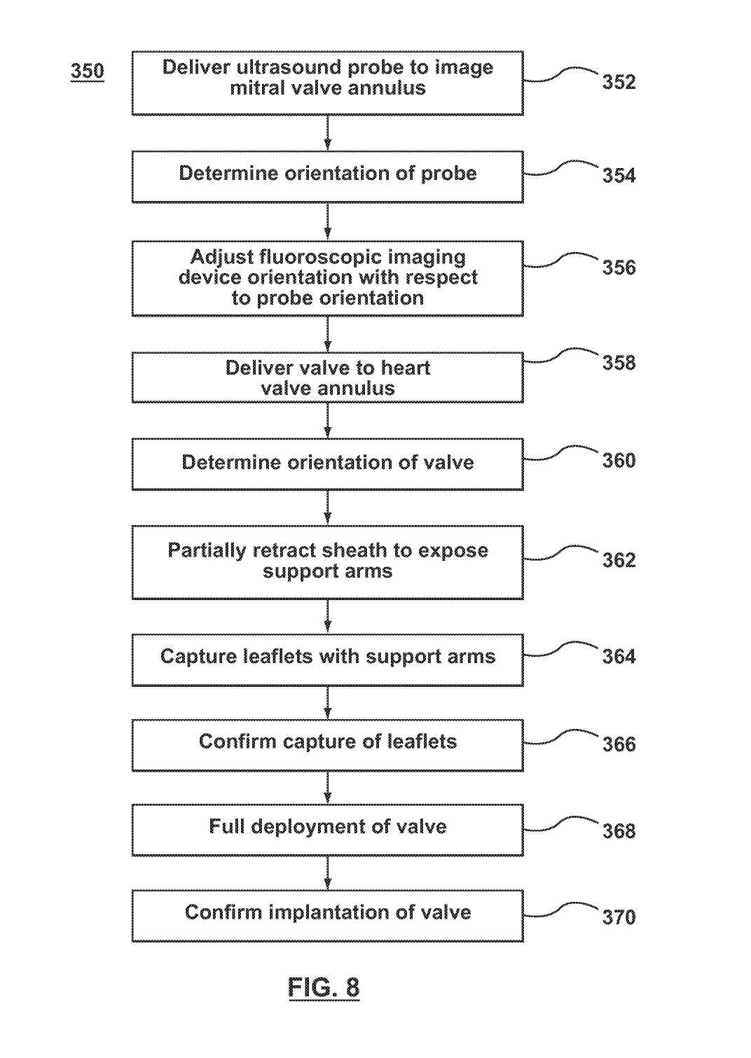

FIG. 8 is a flow diagram of a method 350 for implanting a prosthetic mitral valve 100 as discussed above with respect to FIGS. 5-7. At step 352, the ultrasound probe 40 is delivered to image the mitral valve annulus 300. Next, at step 354, the orientation of the probe 40 is determined using the accelerometer 44 coupled to the ultrasound catheter 42. At step 356, the fluoroscopic imaging device orientation is adjusted with respect to the probe orientation, for example using accelerometer 32. At step 358, the valve 100 is delivered to the mitral valve annulus 300. The orientation of the valve 100 can be determined at step 360 utilizing the accelerometer 56 coupled to the delivery system 50. Next, at step 362, the capsule 150 is partially retracted to expose the support arms 108. The support arms 108 are then positioned to capture the leaflets at step 364. This capture can be confirmed at step 366, for example by evaluating the native valve leaflets. If desired, capture of the leaflets can further be accomplished by simultaneously viewing ultrasound images and fluoroscopic images to “clock” deployment of the support arms 108 with movement of the native leaflets. Once it is confirmed that the support arms have captured the native leaflets, full deployment of the stent can be provided at step 368. At step 370, the ultrasound imaging probe 40 and fluoroscopic imaging device 12 can be used to confirm that implantation has been achieved and that the prosthetic valve 100 is operating properly.

Although the present disclosure has been described with reference to preferred embodiments, workers skilled in the art will recognize that changes can be made in form and detail without departing from the spirit and scope of the present disclosure.

What is claimed is:

1. A method of controlling deployment of a prosthetic heart valve having a frame and opposed support arms connected to the frame, comprising:
positioning an ultrasound imaging probe at a selected position with respect to a target site including a valve annulus;
sensing a probe orientation of the ultrasound imaging probe with respect to the target site at the selected position;
aligning a fluoroscopic imaging device to an aligned orientation with respect to the probe orientation such that an imaging plane for the ultrasound imaging probe is substantially perpendicular to an imaging plane for the fluoroscopic imaging device;
obtaining an ultrasound image of the target site with the ultrasound imaging probe in the selected position;
obtaining a fluoroscopic image of the target site with the fluoroscopic imaging device in the aligned orientation;
determining a valve orientation and a valve position of the support arms with respect to the valve annulus as a function of the ultrasound image and the fluoroscopic image.

2. The method of claim 1, further comprising:
partially deploying the support arms of the prosthetic heart valve to capture leaflets associated with the valve annulus.

3. The method of claim 2, further comprising:
obtaining a second ultrasound image of the target site after partial deployment of the support arms;
confirming the leaflets are captured by the support arms with the second ultrasound image.

4. The method of claim 3, further comprising:
fully deploying the prosthetic heart valve upon confirming that the support arms have captured the leaflets.

5. The method of claim 1, wherein sensing the probe orientation includes coupling an accelerometer to the ultrasound imaging probe to determine the probe orientation with respect to gravity.

6. The method of claim 1, wherein determining the valve orientation includes using an accelerometer to determine an orientation of the support arms with respect to gravity.

7. The method of claim 1, further comprising:
coupling an accelerometer to the fluoroscopic imaging device to determine an orientation of the fluoroscopic imaging device with respect to gravity.

8. A system, comprising:
a fluoroscopic imaging device mounted to a support head maintaining the fluoroscopic imaging device in a selected orientation;
an ultrasound imaging probe including an accelerometer measuring a probe orientation of the ultrasound imaging probe with respect to gravity;
a processor coupled to the accelerometer and providing a signal indicative of the probe orientation relative to the selected orientation.

9. The system of claim 8, wherein the fluoroscopic imaging device further includes a second accelerometer measuring the selected orientation with respect to gravity.

10. The system of claim 8, further comprising:
a delivery device configured to deploy a stented prosthetic heart valve to a target site, the fluoroscopic imaging device and ultrasound imaging probe configured to determine a position of the prosthetic heart valve with respect to the target site.

11. The system of claim 10, wherein the delivery device includes a second accelerometer measuring an orientation of the prosthetic heart valve with respect to gravity.

12. A delivery device for percutaneously deploying a stented prosthetic heart valve including a stent frame to which a valve structure is attached, the device comprising:
a delivery sheath assembly terminating at a distal end and defining a lumen;
an inner shaft slideably disposed within a lumen and in contact with the stent frame; and
an accelerometer providing a signal indicative of an orientation of the stent frame with respect to gravity,
wherein the device is configured to provide a loaded state in which the delivery sheath assembly retains the stented prosthetic heart valve over the inner shaft and a deployment state in which the distal end of the delivery sheath assembly is withdrawn from the prosthetic heart valve to permit the prosthetic heart valve to release from the inner shaft assembly.

13. The delivery device of claim 12, wherein the stent frame includes opposed support arms extending therefrom and further wherein the orientation is indicative of a position of the support arms with respect to gravity.

14. The delivery device of claim 13, wherein the device is further configured to provide a partially deployed state, wherein the delivery sheath assembly is withdrawn from the prosthetic heart valve to expose the support arms.

15. The delivery device of claim 12, further comprising:
a handle connected to the inner shaft assembly; and
wiring connected to the accelerometer and extending from the accelerometer to the handle.

16. The delivery device of claim 12, wherein the accelerometer is one of piezoelectric, piezoresistive and capacitive.

17. The delivery device of claim 12, wherein the accelerometer is triaxial.

18. The delivery device of claim 12, further comprising:
a retainer connected to the inner shaft assembly, wherein the accelerometer is mounted to the retainer.

19. A delivery device for positioning an implantable medical device, comprising:
a catheter including a coupling mechanism for attachment of the medical device; and
an accelerometer coupled to a distal end of the catheter and configured to provide a signal indicative of orientation of the medical device with respect to gravity.

20. The delivery device of claim 19, further comprising wiring coupled to the accelerometer and positioned along the catheter.
